(19) United States
(12) Plant Patent Application Publication
(10) Pub. No.: US 2004/0003442 P1
(43) Pub. Date: Jan. 1, 2004

(54) Photinia plant named 'Cassini'

(76) Inventor: Robert J. Tancredi, Salem, OR (US)

Correspondence Address: BURNS, DOANE, SWECKER & MATHIS, L.L.P., P.O. Box 1404, Alexandria, VA, 22313-1404, US

(21) Appl. No.: 10/185,046

(22) Filed: Jul. 1, 2002

(51) Int. Cl. A01H 5/00

(52) U.S. Cl. PLT/226

(57) ABSTRACT

A new and distinct highly ornamental variety of Photinia×fraseri plant is provided. An upright outwardly-spreading shrub is described that has a more compact growth habit than commonly is displayed by Photinia×fraseri. Attractive variegated foliage is displayed. Initially the foliage is reddish with pink margins that changes to green with pink margins. Finally the leaf margins mature to near white with near white and silver splashes over the green median upper surfaces of the leaves. The new variety propagates well by the use of stem cuttings and grows well in U.S.D.A. Hardiness Zone Nos. 7 and 8. The plant is well suited for growing as an ornamental specimen or in the form of a distinctive hedge. Also, the unusual colorful foliage can be used to advantage as a part of flower arrangements.

### BOTANICAL/COMMERCIAL CLASSIFICATION

Photinia×fraseri/Photinia Plant

### VARIETAL DENOMINATION

cv. ‘Cassini’

### SUMMARY OF THE INVENTION

The invention relates to a new and distinct selection of Photinia×fraseri, a member of the rose family, Rosaceae.

The new variety of the present invention was discovered during 1991 while present in a block of container-grown Photinia×fraseri plants (non-patented in the United States) that had been reproduced through the use of cuttings at Salem, Oreg. A single plant of the new variety was discovered and was carefully preserved and studied in view of its distinctive combination of characteristics. The new variety of the present invention is believed to be a spontaneous whole plant mutation of unknown causation. Had the new variety not been discovered and preserved it would have been lost to mankind.

It was found the new Photinia×fraseri variety exhibits the following combination of characteristics:

(a) an upright outwardly-spreading broad-leaved evergreen shrub that has a more compact growth habit than commonly is displayed by Photinia×fraseri,

(b) forms distinctive variegated foliage that is initially reddish with pink margins, changing to green with pink margins, and with the margins finally maturing to near white with near white and silver splashes over the green median upper surfaces of the leaves,

(c) propagates well by the use of stem cuttings, and

(d) is well suited for providing attractive ornamentation in the landscape.

The new variety can be readily distinguished from its Photinia×fraseri parent which lacks the distinctive variegated foliage and the more compact growth habit.

The new variety well meets the needs of the horticultural industry and can be grown in the landscape as a colorful specimen plant or to form a hedge. The new variety also can serve as a source of unusual colorful cut foliage for use in flower arrangements.

Asexual reproduction of the new variety by the use of stem cuttings at Salem, Oreg. and at West Grove, Pa. has demonstrated that the unique combination of characteristics of the new variety is firmly fixed and is retained through successive generations of asexual propagation.

The new variety of the present invention has been named ‘Cassini’. ‘Cassini’ has not been observed under all possible environmental conditions to date. Accordingly, it is possible that the phenotype may vary somewhat with variations in the environment, such as temperature, light, day length, etc.

### BRIEF DESCRIPTION OF THE PHOTOGRAPHS

The accompanying photographs show, as true as is reasonably possible to make the same in color illustrations of this character, typical plants of the new variety. The plants were approximately five years of age and were being grown in containers outdoors at Salem, Oreg.

FIG. 1 shows primarily the typical distinctive mature foliage of the new variety. Such leaves are shown to display variegated upper leaf surfaces that are green with irregular near white margins and splashes of white and silver. Some immature reddish foliage also is present primarily at the upper left portion of the photograph.

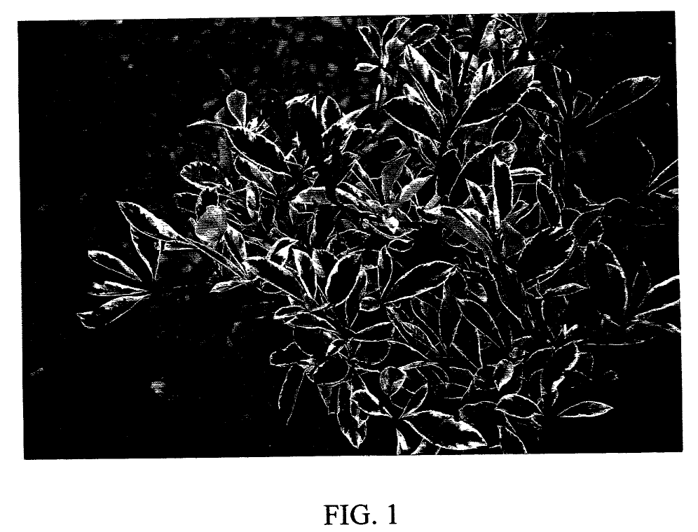

FIG. 2 shows at the forefront the typical colorful immature reddish foliage, as well as the variegated leaves of advancing maturity with pink margins and some overlay white and silver splashes.

### DETAILED DESCRIPTION

The description is based upon the observation of five year-old plants propagated by the use of stem cuttings that were growing outdoors in containers at West Grove, Pa. The chart used in the identification of the colors is the R.H.S. Colour Chart of The Royal Horticultural Society, London. Other color terminology used in the description is to be accorded its ordinary dictionary significance.

Plant:

Type.—perennial, broad-leaved evergreen shrub.

Growth habit.—upright and outwardly-spreading and more compact than its Photinia×fraseri parent.

Branching habit.—freely-branching, and responds well to pruning.

Foliage:

Bearing.—simple and alternate.

Configuration.—mostly oblanceolate.

Apex.—abruptly acuminate.

Base.—narrowly cuneate.

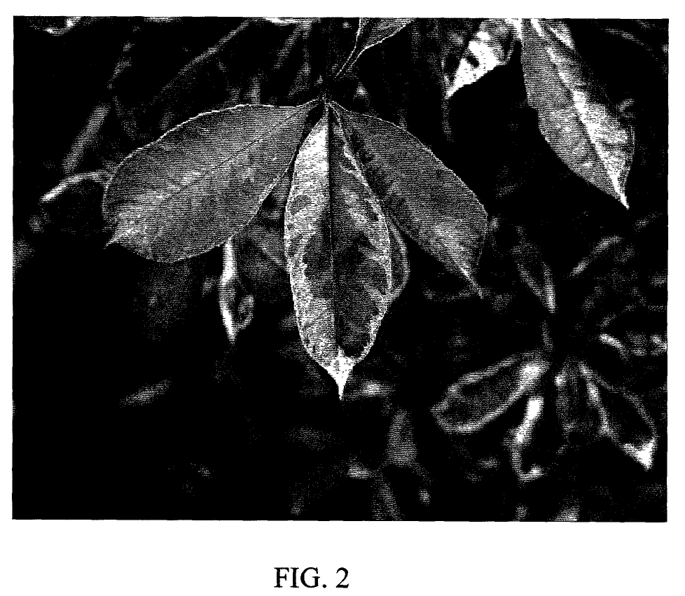

Margins.—serrulate (as illustrated in FIG. 2).

Length.—commonly approximately 5 to 14 cm, with mature leaves frequently measuring approximately 10 to 12 cm in length.

Width.—commonly 2.5 to 5.5 cm, with mature leaves frequently measuring approximately 3.5 to 4.5 cm in width.

Immature foliage ventral surface.—margins: mixture of Red-Purple Group 58D, Red-Purple Group 63C, and Red-Purple Group 65A. — medial areas: primarily Green Group 139A with small scattered areas of various shades near Greyed-Green Group 189B with some of the same margin coloration being scattered throughout and overlaying in a random array of shapes and sizes.

Immature foliage dorsal surface.—the base coloration is near Greyed-Green Group 194B and is overlaid with Greyed-Purple Group 186B.

Mature foliage ventral surface.—margins: well-defined and mostly near Yellow-White Group 158B. medial areas: near Green Group 139A with randomly scattered areas of various shapes near Greyed-Green Group 189A and 189B.

Mature foliage dorsal surface.—mostly near Greyed-Green Group 191B with near white margins near Yellow-White Group 158B.

Petiole.—configuration: commonly grooved on the ventral surface. — length: commonly approximately 0.5 to 1.1 cm in length. — color of ventral surface: mostly Green Group 139A in the groove, and shading into and near Greyed-Purple Group 184A. — color of dorsal surface: mostly near Yellow-Green Group 147D, and shading into and near Greyed-Purple Group 184A.

Stems.—Young stems on current season's growth are near Red Group 46A in coloration.

Flowers:

Size.—Small.

Color.—White.

Fruit.—None observed to date.

Hardiness: Performs well in U.S.D.A. Hardiness Zone Nos. 7 and 8.

Disease/Pest resistance: Generally comparable to the Photinia×fraseri parent during observations to date.

I claim:

1. A new and distinct Photinia×fraseri plant having the following combination of characteristics:
(a) an upright outwardly-spreading broad-leaved evergreen shrub that has a more compact growth habit than commonly is displayed by Photinia×fraseri,
(b) forms distinctive variegated foliage that is initially reddish with pink margins, and with the margins finally maturing to near white with near white and silver splashes over the green median upper surfaces of the leaves,
(c) propagates well by the use of stem cuttings, and
(d) is well suited for providing attractive ornamentation in the landscape;
substantially as herein shown and described.
